(19) United States
(12) Patent Application Publication
(10) Pub. No.: US 2010/0303883 A1
(43) Pub. Date: Dec. 2, 2010

(54) POLYMERIC DRUG DELIVERY SYSTEMS AND THERMOPLASTIC EXTRUSION PROCESSES FOR PRODUCING SUCH SYSTEMS

(75) Inventors: Wayne C. Pollock, Lincoln University, PA (US); Stuart A. Grossman, Towson, MD (US); Albert H. Owens, Bel Air, MD (US)

Correspondence Address: NIXON & VANDERHYE, PC, 901 NORTH GLEBE ROAD, 11TH FLOOR, ARLINGTON, VA, 22203, US

(73) Assignee: AXXIA PHARMACEUTICALS, LLC, Bel Air, MD (US)

(21) Appl. No.: 12/738,113

(22) PCT Filed: Oct. 17, 2008
(86) PCT No.: PCT/US2008/011908

§ 371 Date: Aug. 20, 2010

Related U.S. Application Data
(60) Provisional application No. 60/960,853, filed on Oct. 17, 2007.

(51) Int. Cl. A61F 2/00; A61K 31/485; A61P 29/00; B05D 3/00

(52) U.S. Cl. 424/423; 514/282; 427/2.14

(57) ABSTRACT

Implants are disclosed for delivery of therapeutic agents such as opioids and the manufacture and uses of such implants.

### FIELD OF THE INVENTION

The subject invention relates to implants for delivery of therapeutic agents such as opioids, and the manufacture and uses of such implants.

### BACKGROUND OF THE INVENTION

U.S. Pat. Nos. 5,633,000, 5,858,388, and 6,126,956 to Grossman et al. relate to drug delivery systems containing an active agent such as an opioid. These implants have a geometry such that the release of the active agent is continuous over extended periods of time. The patents also relate to the manufacture and various uses of the implants.

The polymeric implant delivery system described in U.S. Pat. No. 6,126,956, issued to Grossman et al, discloses a blend of the active compound with Elvax 40W when fabricated. The thickness, diameter and central channel surface area, provide the release kinetics and blood level required for therapeutic benefit.

Grossman et al teach a solvent based process for producing both the internal drug reservoir matrix as well as the drug impermeable external coating (e.g. (poly)methylmethacrylate). Such a process is difficult to automate, slow and expensive due to the time it takes to dry and remove the solvent(s) and also because of the cost of the organic solvents which have no actual value in the finished product. There is also a risk that retained solvent volatiles in the implant could result in cytotoxicity in the final product.

Hot-Melt Extrusion (HME) of drug delivery systems, including implants, offers many advantages over traditional pharmaceutical manufacturing processes. Neither solvents nor water are required. Fewer processing steps are needed, time consuming drying steps are eliminated and drug degradation due to hydrolysis is not an issue.

With HME, one or more active drug substances in powder or granular form can be dry blended with one or more thermoplastic polymers possibly including certain functional excipients, enhancers and plasticizers. During advanced technology pharmaceutical hot melt extrusion processes, these material components are precisely measured and introduced by a computer controlled gravimetric feeding system into the hopper and then into the feed or mixing section of the extruder barrel. The powders are mixed and transformed into a homogeneous molten matrix by the shearing, frictional action of the screw and by heating zones within the barrel of the extruder.

A schematic diagram of a single screw hot melt extruder is provided in FIG. 1.

A more sophisticated GMP, twin screw pharmaceutical extruder can be used in the case of a fully integrated, single step manufacturing process. Such an extruder is exemplified by the loop controlled, 600 rpm, 25 hp Leistritz ZSE-27 mm twin screw melt compounding unit.

### SUMMARY OF THE INVENTION

The subject invention relates to a subcutaneous delivery system comprising: a biocompatible thermoplastic polymer matrix, a therapeutic agent embedded homogeneously in said matrix, and a biocompatible drug impermeable thermoplastic polymer coating said matrix, wherein said delivery system has a geometry such that there is an external coated wall and an internal uncoated wall (or channel) forming an opening for release of said therapeutic agent, and the distance between the uncoated wall and the coated wall opposite the uncoated wall is substantially constant throughout the delivery system. In an advantageous embodiment, the therapeutic agent is hydromorphone which is present at greater than 40 or 50% of the polymer matrix, which advantageously also includes EVA.

The invention also relates to a method of producing a subcutaneous implant comprising the steps of i) forming a matrix polymer sheet by hot melt compounding a first thermoplastic polymeric resin with a therapeutic agent, ii) die cutting said sheet to form polymer matrix, and iii) coating said polymer matrix with a second thermoplastic polymeric resin. In another embodiment, the subcutaneous implant delivery system having an uncoated central channel is produced by co-extruding of a first thermoplastic polymeric resin and a therapeutic agent and a second thermoplastic polymeric resin into a multiple cavity die to form a coated polymer matrix.

The invention also includes a method of providing prolonged relief of pain in a mammal suffering from pain comprising subcutaneously administering the subcutaneous delivery system described above.

### BRIEF DESCRIPTION OF THE DRAWINGS

Reference is made to the accompanying drawing Figures, wherein:

FIG. 1 is a schematic diagram of a single screw extruder;

FIG. 2 is a diagram showing the implant diensinos chose for the study described in greater detail below;

FIG. 3 shows the injection nozzle used to transfer molten polymer from the melt plastometer to the molds in the study;

FIGS. 4A and 4B show the injection base of the injection mold;

FIG. 5 shows the injection mold containing vented disk-shaped reservoirs;

FIG. 6 is a graph of the amount of hydromorphone hydrochloride n ug/hr released from coated disks of 50% hydromorphone hydrochloride and 50.0% Evatane® 28-800 with various dimension over eight days;

FIG. 7 is a graph of the amount of hydromorphone hydrochloride in ug/hr released from different grades of Evatane® disks with 50% hydromorphone hydrochloride over eight days. Dimension of all disks were 12.6×2.7 mm;

FIG. 8 is a graph of the amount of hydromorphone hydrochloride in ug/hr released from coated 12.6×2.7 mm disks of different concentrations of hydromorphone hydrochloride and Evatane® over eighteen days;

FIG. 9 is a graph of the amount of hydromorphone hydrochloride in ug/hr released from coated 12.6×2.7 mm disks containing polyethylene glycol, hydromorphone hydrochloride and Evatane® 28-420 over six days;

FIG. 10 is a graph of the amount of hydromorphone hydrochloride in ug/hr released from coated 10.5×2.7 mm disks of different concentrations with micronized hydromorphone hydrochloride and Evatane® over five days; and

FIG. 11 is a graph which shows that the dissolution rate levels out after the burst on the 2^{nd} day while at 1-month, approximately 90 mg of hydromorphone HCl is released of the 300 mg in the implant.

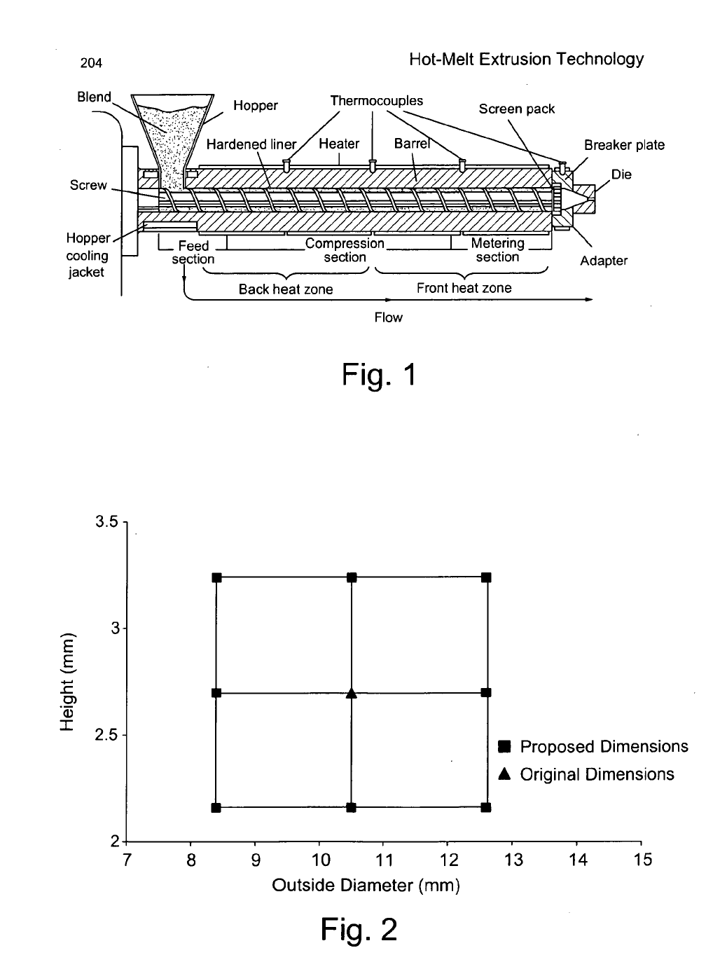

### DETAILED DESCRIPTION OF THE INVENTION

The subject invention relates to implant devices that permit controlled release of a therapeutic agent by subcutaneous implant. The devices provide burst free systemic delivery with near constant release of an active agent for a long duration, i.e. greater than 2 weeks, greater than 4 weeks, greater than 8 weeks, greater than 12 weeks, greater than 16 weeks or greater than 6 months. In specific embodiments of the device, more than one drug can be delivered where the delivery of both drugs is systemic, or the delivery of one drug is systemic without burst while the delivery of the other is local with or without burst.

The geometry, manufacture and use of implants are disclosed in commonly owned U.S. Pat. No. 5,858,388, hereby incorporated by reference in its entirety. In a new embodiment, one or more openings are added to the perimeter wall of cylindrical, eg disk, implants.

Polymeric drug delivery devices in the form of a subcutaneous implant for reservoiring and controlled steady state release of therapeutic agents such as opioids including hydromorphone, can utilize several categories of thermoplastic resins for:

i) the drug reservoir controlled release matrix, and/or
ii) the drug impermeable coating

The present invention relates to implants made with hot-melt extrudable, thermoplastic polymers, and to processes including dry blending, hot melt compounding and extrusion for manufacturing the implant. The processes of the invention are solvent free, potentially fully integrated, melt blending, compounding, extrusion/co-extrusion and molding processes which provide the capability to manufacture the entire multi-component implant in a single, digitally monitored and controlled operation.

Key functional benefits which accrue from the use of hot melt extrudable thermoplastics and melt processing in the manufacture of the implant are the improved impact resistance and resistance to cracking that can occur with the hard and brittle coatings e.g. (poly)methylmethacrylate, as the external drug impermeable coating. The coating, the purpose of which is to restrict the release of drug to the surface area of uncoated polymer in the central channel, allows uniform controlled flux with no burst effect. The coating is a significant factor in preventing possible leakage of the active opioid and a potentially uncontrolled and lethal burst effect while the implant is in use. Co-extrusion enables i) multi-layer external polymer construction, insuring against leaks due to pinholes, ii) the manufacture of a multi-layer composite external polymer wherein a specific polymeric drug barrier is included in the structure-insuring against uncontrolled diffusion of active resulting in a burst effect during use, and iii) the manufacture of a multi-layer composite external polymer including a specifically selected adhesive tie coat to secure and optimize physical and structural integrity of the implant by enhancing the bond between components.

Examples of thermoplastic resins useful for i) the drug reservoir matrix and ii) the impermeable coating include:

### Unmodified Homopolymers

Low-density polyethylene
Linear low-density polyethylene
Amorphous polypropylene
Polyisobutylene

### Copolymers

Especially important are copolymers of ethylene.
   Ethylene Vinyl Acetate (EVA) up to 40% VA content
   Ethyl Acrylate (EAA). Ethylene Acrylic Acid resins
   Ethylene Methacrylate (EMA)
   Ethylene ethyl acrylates (EEA)
   Ethylene butyl acrylate
   Thermoplastic Polyurethanes-including but not limited to resins based on:
      Toluene Diisocyante (TDI)
      Methylene diisocyante (MDI)
      Polymeric isocyantes (PMDI)
      Hydrogenated methylene diisocyante
   Thermoplastic copolyesters; eg, DuPont Hytrel
   Thermoplastic Nylon Copolymers; eg, PEBAX
   Thermoplastic Acrylic hydrogels
   Thermoplastic Urethane hydrogels
   Polyethylene Oxide hydrogen

### Thermoplastic Resin Blends

It is possible to create a unique polymeric matrix in which to compound hydromorphone by blending combinations of the above polymers and copolymers. A simple example is utilizing selected molecular weights and variations within the same basic ethylene vinyl acetate (EVA) resin category. These resins are available commercially as DuPont Elvax. Any one or combination of these grades and percent combinations of resins, functional excipients, plasticizers with various loadings of active drug substance provide the formulator with a wide set of possibilities for controlling drug delivery parameters.

| ELVAX GRADE | % VINYL ACETATE |
|---|---|
| 40W | 40 |
| 150 | 32 |
| 265 | 28 |
| 360 | 25 |
| 460 | 18 |
| 660 | 12 |
| 760Q | 9 |

In order to optimize a resin blend in terms of compatibility, it is advantageous to select resins within the same category of polymers or copolymers, and combine these in such a way as to modify solubility or dispersion of the selected drug substance, e.g. hydromorphone HCl, in the polymeric matrix. Relative solubility and dispersion uniformity of the active pharmaceutical compound in the polymeric resin blend are factors influencing drug delivery rate or flux from the subcutaneous implant. This blending of the reservoir polymers and the use of excipients and plasticizers provides one means for controlling drug delivery rates while optimizing other functional properties such as hydrolytic stability, drug loading capacity, drug compatibility and biocompatibility. Additionally, such custom formulation and blending of thermoplastic resins, plasticizers and excipients allows the optimization of critical physical properties which important in the final product including tensile, modulus, crack and friability resistance, impact resistance and elongation.

Commercial versions of the above polymers, are readily available, as shown by the above example of a series of resins in the Elvax line of EVA resins. These can be dry blended and melt compounded together with excipients and/or plasticizers along with the active drug substance using single or twin screw hot melt extruders to create a delivery system for controlled release of the drug. These custom blended hot melt extrudable formulations are highly amorphous (excellent drug compatibility and high loading capability), relatively low melting feedstock systems which will process using extrusion, compounding and injection molding techniques without subjecting the drug to temperatures which may cause decomposition and loss of therapeutic efficacy.

Release kinetics from a melt blended and extruded polymeric matrix are a function of the drug components and loading, the polymer types, polymer morphology (Tg) and additives including excipients and plasticizers. A skilled person in the art can select the appropriate polymer or polymer blend and additives (e.g. excipients) to achieve the desired therapeutic blood level of for a given active agent.

For a different active drug or combination of drugs, or different therapeutic indications in human or animal subjects, the skilled person will specify a different set of release kinetics. It is possible to select from a series of polymeric resins or resin blends to achieve the desired kinetics and optimum therapeutic blood levels for specific human or animal indications for hydromorphone and other selected drugs or combinations of drugs.

Examples of formulations are:
Formulation 1
50% Hydromorphone HCl
50% Elvax 40W
Formulation 2
50% Hydromorphone HCl
25% Elvax 40W
25% LDPE (low density polyethylene)
Formulation 3
50% Hydromorphone HCl
12% Elvax 40W
38% LDPE
Formulation 4
50% Hydromorphone HCl
50% LDPE
It should be understood that Elvax 40 W is just one example. Other resins or resin blends as listed above can be used depending on the specific drug(s), the loading, delivery rate or duration of activity required. Those resins include any one the lower vinyl acetate conataining grades of Elvax listed above, the ethylenic copolymers listed as well as the thermoplastic copolyesters, Nylon copolymers and thermoplastic polyurethanes.
Any of these resins or resin blends can be hot melt compounded with hydromorphone HCl at various loadings up to 50% to create the internal matrix (reservoir component) of a drug delivery implant with the flux and duration of therapeutic activity required.

### Thermoplastic Polyurethanes

Tecoflex Medical Grade Thermoplastic Polyurethanes (Grades EG-80A, EG-93A and EG-60D comprise a group of aliphatic, polyether based resins that have establish credentials for implants including having passed the following standard screening tests: MEM Elution, Hymolysis, USP Class VI, 30 Day Implant, and Ames Mutagenicity.

These urethane resins have been evaluated in several medical device applications that involve the requirement for high permeability to moisture vapor. They are highly amorphous compounds which allows them to be used for drug delivery systems where high loading and flux rate are required.

Tecoflex EG-80 and Tecoflex EG-85 are both made from the same diisocyante (HMDI) and the same 2000 molecular weight PTMEG polyol but the ratios of polyol to diisocyante (hard segment to soft segment) are different. The lower modulus, lower Tg version—Tecoflex EG-80—is more amorphous and less crystalline in its morphology resulting in a higher flux drug delivery formulation. Tecoflex EG-60 is based on the same HMDI diisocyante but a 1000 molecular weight PTMEG polyol, resulting in a different morphology, crystallinity and drug flux.

A series of specific formulations can be made using various combinations of the above Tecoflex resins.

Other thermoplastic polyurethanes including Tecoflex EG-85, EG-93A or EG-60D can be used alone or blended together with hydromorphone HCl or other drugs to form the feedstock for the internal polymer matrix. It is to be understood that ratio of drug to polymer is variable within the scope of this invention.

Polymer blends can include two or more resins within the same category of resins; eg, Elvax 40W with Elvax 460 and Elvax 660. These blends can also include polymers from different categories; eg, ELVAX 40W and Tecoflex EG-85.

The drug impermeable coating is advantageously selected from the series ethylene vinyl acetate thermoplastic resins including but not limited to Elvax E-40 with the core reservoir polymer for the extended release analgesic component; eg, hydromorphone HCl being selected from the same family of ethylenic copolymers. Another advantageous implant structure utilizes one of a series of medical and pharmaceutical ether type thermoplastic polyurethane resins based on either hydrogenated methylene diisocyante (HMDI) or methylene diisocyante (MDI) listed above as the hard segment of the polymer and either polyethylene glycol (PEG) or polytetramethylene ether glycol (PTMEG) as the soft segment.

While these EVA and thermoplastic polyurethane polymers are advantageous, any of the copolyesters, Nylon copolymers or ethylenic copolymers listed above can be used alone or as resin blends to form the internal or external polymeric components of the implant.

### Biodegradable Implants

Like the non-biodegradable implants disclosed above the biodegradable implants of the invention provide burst free systemic delivery, near constant release for a long duration. The geometry of these devices is the same as the non-biodegradable implants described above but they are manufactured with biodegradable materials. In an advantageous embodiment, the biodegradable interior disintegrates faster than the biodegradable external polymer. In another embodiment, one can use radiofrequency or ultrasonic ablation of empty polymer obviating need for removal.

In another embodiment, the implant achieves systemic delivery, burst free, constant release, long duration like the implants above, but also allows the insertion of the implants without surgical intervention (ie needle or trochar). The implants are of a size which permits insertion by a needle or trochar. The implants utilize different coatings and/or internal polymers that release similarly to time release capsules.

### Functional Excipients and Plasticizers

Functional excipients which can be included in the melt blend formulation for either the implant drug reservoir core or drug impermeable coating can be broadly classified as matrix carriers, release modifying agents, bulking agents, foaming agents, thermal stability agents, melt viscosity control materials, lubricating agents or adhesion promotion agents and primers for enhancing core to coating integrity. Functional excipient materials for hot melt extrudeable pharmaceutical formulations are in many cases the same compounds used in previous traditional solid dosage forms.

Plasticizers are typically incorporated into thermoplastic resin formulations as process aids to minimize friction or thermal degradation of the active pharmaceutical compound during hot melt extrusion or to modify physical properties in the finished injection molded or fabricated product. The choice of plasticizers to lower processing temperatures depends on several factors including compatibility with the resin system and as well as process and long tem stability. Typical pharmaceutical grade plasticizers for use in hot melt formulations include triacetin, citrate esters along with low molecular weight polyethylene glycols and phthalate esters.

One particularly useful functional excipient is supercritical CO2 which is advantageously injected at controlled temperature and pressure (e.g. approximately 40 degrees C. and 1000 PSI) into the melted polymer through a downstream port in the extruder barrel as disclosed in US Patent Application 20050202090 hereby incorporated by reference in its entirety. In the subject invention involving an extended release subcutaneous polymeric implant for systemic delivery of analgesics including hydromorphone HCl, the active agent is dry blended between 10% and 90% by weight with a polymeric resin or resin blend, advantageously an implant grade TPU (thermoplastic polyurethane) such as Polymer Technology Group Elasthane 80 A or a high vinyl acetate content EVA such as Arkema Evatane 28-420. This uniformly dry blended feed stock is introduced into the hopper of a twin screw extruder where it is melt compounded into a liquid mass which upon cooling is pelletized and in turn used as a feedstock for an injection molding process which produces the three dimensional implant device.

During the molding process, supercritical liquid CO2 is injected through a port in the equipment into the molten drug/polymer matrix under the elevated temperature and pressure conditions specified herein. These conditions maintain the supercritical CO2 in liquid form forming a single phase solution with the polymer. The supercritical CO2 dissolves in the polymer. As the molten matrix of active drug/polymer and excipient are fed into the mold, the material is controllably cooled resulting in a thermodynamically unusable system causing the excipient to revert to gaseous form where it is nucleated by the uniform drug particle size and content to form bubbles which on final cool results in an interconnecting microcellular structure or foam.

In addition to reducing the temperature required to achieve optimum melt viscosity for extrusion thereby reducing the impact of thermal degradation on the active drug substance, this gaseous material creates controlled porosity and interconnecting cellular structure in the polymeric matrix which significantly increases the surface area of drug loaded polymer available for contact by body fluids, thereby enhancing dissolution and delivery of the active to systemic circulation.

More specifically, the functional benefits created by such a interconnecting cellular drug/polymer matrix are: i) improved access for body fluids from subcutaneous implant site into the core of the drug reservoir for more complete dissolution, ii) reduced retained active in the implant thus reducing the possibility of recovery and illicit use, iii) increased surface area for dissolution which maximizes delivery to systemic circulation, iv) improved uniformity of delivery which minimizes the possibility of uncontrolled burst effect.

Other well known blowing agents including nitrogen generating materials can be utilized in the process of the invention.

### Radio-Opaque Markers

Radio-opaque pigments; e.g., TiO2, can be conveniently melt blended in either or both exterior or interior polymers enabling the implant to be easily located by X-ray in the event removal is required or useful. Other imbedded markers have the potential of providing important information about the implant once in place in the patient including dose in ug/hr, expected duration of release of the active analgesic (hydromorphone HCl) and date of implantation. Such information can be linked to a database available to physicians.

### Implant Manufacturing Processes

### Hot-Melt Compounding and Extrusion

Hot-Melt Extrusion (HME) of drug delivery systems including oral, transdermal and implant dosage forms has been well established in the industry and offers many advantages over traditional pharmaceutical manufacturing processes. Neither organic solvents nor water is required-resulting in substantial materials and process cost savings. Fewer processing steps are needed. Time consuming and expensive drying steps are eliminated. Drug degradation due to thermal stress or hydrolysis are removed as issues along with the toxicity risk resulting from retained organic volatiles.

Hot-melt compounding and extrusion using advanced co-extrusion techniques provides the opportunity to produce sophisticated multi-layer and multi-functional composites by creating and bringing together several melt streams in a single fully integrated manufacturing process. This provides the option of creating a device with one or more active drug substances dispersed in one or more polymeric matrices as well as the ability to design pharmaceutically inert functional members such as rate controlling membranes, structural components, adhesive tie layers and drug impermeable barrier composites.

In the case of producing drug/polymer matrices, one or more active drug substances in powder or granular form can be dry blended with selected polymers or polymer blends along with functional excipients and plasticizers. These materials are introduced by computer controlled gravimetric feeding systems into the extruder/compounder where they are transformed in to a homogeneous molten matrix by the shearing frictional action of the screw and heating zones within the barrel of the extruder. It is also possible to introduce additional functional excipients including but not limited to the preferred gaseous plasticizer and foaming agent, supercritical C02, into the melted polymer through a downstream injection port in the extruder barrel. The finished melt compound drug/polymer blend is finally pushed by the action of the turning screw though a die section attached to the end of the extruder where it is either cooled, chopped into small cylinders or pelletized into a feed stock for a subsequent hot melt process which molds the final product. Advantageously, all of these steps can be consolidated into a single fully integrated and automated process beginning with compounding and ending with an injection molding process which produces the drug delivery system.

Hot melt extrusion equipment consists of an extruder, downstream auxiliary equipment and monitoring tools used for process control. The extruder is typically composed of a feeding hopper, barrel, screw, die, power unit to drive the screw along with heating and cooling equipment. Also included are temperature gauges, screw speed controller, extrusion torque monitor along with pressure gauges. Depending on whether the melt goes directly into a molding operation or into pellets or granules for a secondary process, such down stream hardware is included in the hardware sequence.

In one embodiment of the pharmaceutical melt blending process, the molten drug/polymer matrix can be directly formed into the final implant specifically consisting of a core or matrix of hydromorphone HCl, melt blended with one or more polymeric resins or resin blends, optionally with excipients or plasticizers, together acting as a binder and drug reservoir. The drug impermeable outer coating is also applied along with the central uncoated channel-all in one continuous operation.

The resins, resin blends, functional excipients, enhancers, plasticizers and optionally radio-opaque additives can be i) mixed and dry blended together along with an active agent such as hydromorphone for the reservoir matrix or ii) combined without active drug for the impermeable outer coating. Dry blended formulations for either matrix or coating can be subsequently utilized as feedstock for a melt compounding and extrusion or co-extrusion process as defined above. The extrudate from the hot melt blending and compounding process can be either i) cooled and collected as pellets for use as feedstock in a film or sheet extrusion process or ii) directly processed by single layer or multi layer film/sheet coextrusion or injection molded into the finished implant.

The drug impermeable coating is hot melt extrusion or coextrusion coated, powder coated and fused, or solution coated using any of the EVA, ethylenic polymers, ethylenic copolymers, copolyesters, Nylon copolymers or thermoplastic polyurethanes listed above either singly or in blends of two or more resins in the same or different polymer categories.

Two advantageous processes can be used separately or in combination to fabricate the final implant:
1. Single layer or multilayer injection molding of reservoir matrix, outer coating or the entire matrix/coating composite with or without central uncoated channel.
2. Single or multilayer sheet extrusion of core component followed by melt, fused powder coating or solution coating of core with outer impermeable layer.

The uncoated central channel is the only area through which the active compound, e.g. hydromorphone HCl can exit the implant. The flux or rate of delivery of the drug substance is directly proportional to and controlled by the exposed surface area in the uncoated central channel. The central channel is advantageously formed as part of the fully integrated hot-melt extrusion and molding process but can also be produced by laser drilling or by perforating the polymer with a precise diameter device.

### Multi-Layer External Drug Impermeable Coating

In an advantageous embodiment, the external drug impermeable coating is composed of two or more layers, for example, each between 24 and 48 microns in thickness. The following options are possible using hot-melt co-extrusion technology:

### Two Layer Impermeable Coating

Two layers composed of the same polymer preferentially including but not limited to copolymers of ethylene and vinyl acetate, and certain aliphatic ether type thermoplastic polyurethanes based on hydrogenated methylene diisocyante (HMDI) or aromatic ether based thermoplastic urethanes based on methylene diisocyante (MDI) as the hard segment of the polymer and polyethylene glycol (PEG) or polytetramethylene ether glycol (PTMEG) as the soft segment. The purpose of this design is to eliminate the possibility of pin holes which if present could result in a lethal burst of the active opioid ingredient in the final product. It is virtually impossible for two pinholes to be coincident, so that if a pinhole forms in one layer of the external coating, it will be covered and eliminated by the second layer. Other polymers or blends of polymers suitable for this application include ethylene acrylate (EAA), ethylene methacrylate (EMA), ethylene ethyl acrylate (EEA), thermoplastic copolyester (Hytrel), thermoplastic polyamides (PEBAX), low density polyethylene (LDPE), linear low density and polyethylene (LLDPE).

### Three Layer Impermeable Coating

Three layers wherein the top and bottom layer are composed of the same polymers disclosed above with a third, centrally placed inter-laminar barrier film sandwiched between them. An advantageous inter-laminar barrier film is selected from certain functional polymers which have been designed and optimized for this diffusion barrier purpose including but not limited to a homopolymer of vinylidene chloride or a copolymer of vinylidene chloride and vinyl chloride. A composite barrier film can also be co-extrusion coated using any of the polymers or polymer blends listed above and laminated in such a way as to include a physical barrier such as aluminum foil. The result is a structural member within the implant delivery system which precludes the possibility of the patient receiving a lethal burst of active opioid analgesic as a result of a leak that compromises the exterior drug impermeable coating (s).

In another embodiment, the internal layer (that which is immediately adjacent to the internal drug reservoir polymer matrix) is selected from a group of polymers which act as an adhesive tie coat to optimize adhesion between the external, drug impermeable coating (s) or composite laminate and the internal polymeric matrix which serves as the drug reservoir. An advantageous adhesive tie coat is based on the ethylenic anhydride (commercially known as Bynel) which can be extruded or coextruded with the thermoplastic polyurethane, ethylene vinyl acetate copolymers as well as all of the polymers identified and listed above. The specific adhesion between all of these polymers and Bynel is extremely high, thus optimizing the structural integrity of the entire implant.

### Multi-Drug Delivery Device

In another embodiment of the invention, an additional drug (or drugs) can be loaded in the polymer matrix with the first drug, or loaded in a second polymer matrix.

### Systemic Delivery

More than one drug can be delivered where the delivery of both drugs is systemic, or the delivery of one drug is systemic without burst while the delivery of the other is local with or without burst.

### Systemic Delivery and Local Delivery

This system includes a component which provides burst free systemic delivery at near constant release for a long duration (as described above). The system also provides a second component for local delivery, with or without burst and with variable delivery duration. Potential drugs for use in the second component are antibiotics, anti-inflammatory drugs and anesthetics.

One embodiment of a multi-layer implant for delivering two drugs (e.g. an anesthetic and an opioid) is detailed below:

1. The outer layer is a rapid release polymer/drug matrix. The polymer can be selected from a series thermoplastic polyurethanes, co-polyesters or copolymers of nylon and polyethylene glycol (PEG) or polytetramethylene ether glycol (PTMEG) which have been optimized in terms of the amorphous structure necessary to insure high flux or rapid delivery of the anesthetic component of

2. The next layer in coming from the outside of the implant is the anesthetic drug reservoir component. The polymer is optimized for compatibility, drug loading capacity and stability with the drug. Advantageous polymers for this component are by category the same ethylenic copolymers and thermoplastics as listed above for the rapid release layer of the device but require the selection of one or more of the more crystalline, less amorphous (lower Tg) resins.

The next layer in, is an impermeable coating which serves to separate the short term anesthetic from the extended release opioid analgesic (e.g. hydromorphone HCl) in the internal drug reservoir matrix That inter-laminar barrier layer is a polymer designed for optimum barrier properties including but not limited to homopolymers of vinylidene chloride or copolymers of vinylidene chloride and vinyl chloride or coextrusion laminates of those Saran type barrier polymer with the ethylene vinyl acetate copolymers, thermoplastic polyurethanes, LDPE, LLDPE, thermoplastic copolyesters (Hytrel) or thermoplastic copolyamides (PEBAX) listed above.

The central core is composed of the extended release analgesic, e.g. hydromorphone HCl, embedded in a polymeric matrix based advantageously on copolymers of ethylene and vinyl acetate or certain thermoplastic aliphatic or aromatic polyether based polyurethanes or the other ethylenic polymers or copolymers or polyester copolymers (Hytrel) or Nylon copolymers as identified above.

This design requires one or more polymeric reservoirs and coatings, For example, the rapid release outer layer matrix for the anesthetic drug component is a highly amorphous, non crystalline thermoplastic polymer such as one of the medical grade aliphatic ether type polyurethanes, while the anesthetic reservoir is another, more permeable resin from the same category of polyurethane polymers to provide a driving force from reservoir to drug delivery layer.

### Uses of the Implants of the Invention

The delivery systems of the invention are useful for delivery of therapeutics for extended periods of time, e.g. 2 weeks to six months.

### Delivery of Opioids

The invention also includes methods of treating pain, e.g. cancer pain, by subcutaneous administration of a delivery system containing an opioid such as hydromorphone. Other opiods useful in the subject invention include morphine analogs, morphinans, benzomorphans, and 4-phenylpiperidines, as well as open chain analgesics, endorphins, encephalins, and ergot alkaloids.

Advantageous compounds, because of their potency, are etorphine and dihydroetorphine which are 1,000 to 3,000 times as active as morphine in producing tolerance to pain (analgesia). 6-methylene dihydromorphine is in this category, also, and is 80 times as active as morphine. Buprenorphine (20-40× morphine) and hydromorphone (perhaps 2-7× as potent as morphine) also belong to this class of compounds. These five compounds, and many more, are morphine analogs.

The category of morphinans includes levorphanol (5× morphine). A compound from this group is 30 times more potent than levorphan and 160× morphine. Fentanyl, a compound that does not follow all the rules for 4-phenylpiperidines, is about 100 times as potent as morphine.

The benzomorphan class includes Win 44, 441-3, bremazocine and MR 2266 (see Richards et al., Amer. Soc. for Pharmacology and Experimental Therapeutics, Vol. 233, Issue 2, pp. 425-432, 1985). Some of these compounds are 4-30 times as active as morphine.

Delivery of Other Active Agents where a Burst is Dangerous

Advantages of the subject delivery system are that it provides systemic delivery, burst free, constant release, long duration. Thus, the system is advantageous for situations where burst might be dangerous—examples are the delivery of anti-hypertensives and antiarrhythmics.

Delivery of Active Agents where Drug is Wasted in Burst

Another situation is where drug is wasted in burst. Examples are: Infectious disease-antibiotics, antivirals, antimalarials, anti-TB drugs, hormones or hormonal blockers, androgens, estrogens, thyroid drugs, tamoxifen, antiseizure drugs, psychiatric drugs, anti-cancer drugs, antiangiogenics, and vaccines.

Delivery of Active Agents where Compliance is Important

The implant is useful in the delivery of active agents where compliance is important such as in the treatment of opioid addiction by administration of methadone or hydromorphone.

### Veterinary Applications

The implants of the subject invention can also be used as noted above for corresponding veterinary applications e.g. for use in delivering active agents to dogs or cats.

The following Examples are illustrative, but not limiting of the compositions and methods of the present invention. Other suitable modifications and adaptations of a variety of conditions and parameters normally encountered which are obvious to those skilled in the art are within the spirit and scope of this invention.

### EXAMPLES

### Example 1

### Internal Polymer—50% Hydromorphone HCl/50% Elvax 40W

A 50% blend of Hydromorphone HCl powder and Elvax 40W pellets or powder is dry blended together with additives as required; eg, plastizers including but not limited to certain low molecular weight polyethylene glycols or radio-opaque pigments including but not limited to TiO2 pigments and subsequently utilized as feedstock for a hot melt compounding and extrusion or co-extrusion process. This formulation will be the drug reservoir matrix component of the finished implant. The exudates from the hot melt blending and compounding process are fed directly to an injection molding or thermal molding process that forms the internal polymeric component in its desired shape and configuration-ready for a sequential series of processes wherein the external drug impermeable coating and uncoated central channel are created.

### Example 1A

### Internal Polymer—50% Hydromorphone HCl/50% Elvax 40W

A 50% blend of Hydromorphone HCl powder and Elvax 40W pellets or powder is dry blended together with additives as required; eg, plastizers. The blended materials are subsequently utilized as feedstock for a hot melt compounding and extrusion or co-extrusion process. This formulation will be the drug reservoir matrix component of the finished implant. The molten mass or cooled, pelletized particles of the polymer/drug blend is fed into a sheet extruder producing a continuous web at the desired thickness of the internal polymer component which after cooling is die cut or mechanically punched in the required diameter of the implant.

### Example 2

### Internal Polymer—50% Hydromorphone HCl/50% Polyurethane; eg, Tecoflex EG-80, a Copolymer of HMDI and a 2000 Molecular Weight PTMEG Polyol

A 50% blend of Hydromorphone HCl is hot melt blended with 50% of a medicinal and pharmaceutical implant grade thermoplastic polyurethane; eg, Tecoflex EG-80, a copolymer of HMDI and a 2000 molecular weight PTMEG polyol.

The external drug impermeable coating is hot melt extrusion or coextrusion coated, using the thermoplastic polyurethane.

### Example 2A

### Internal Polymer—50% Hydromorphone HCl/50% Polyurethane; eg, Tecoflex EG-80, a Copolymer of HMDI and a 2000 Molecular Weight PTMEG Polyol

A 50% blend of Hydromorphone HCl is hot melt blended with 50% of a medicinal and pharmaceutical implant grade thermoplastic polyurethane; eg, Tecoflex EG-80, a copolymer of HMDI and a 2000 molecular weight PTMEG polyol.

The external drug impermeable coating is powder coated and fused, using EVA.

### Example 3

### Method of Manufacture

EVA is commercially available from DuPont and Arkema as pellets that are approximately 1 to 2-mm in diameter whereas Hydromorphone HCl is packaged as a powder. It is not feasible to blend the two materials as purchased without first reducing the particle size of EVA, solvent casting, or by a melt process. Although it is possible to cryogenically grind EVA, this method is prohibitively expensive and does not provide sufficiently small particles.

In one method of manufacture, materials are compounded in a Leistritz twin-screw extruder with dual hoppers. In this process, EVA is fed at the beginning of the extrusion line with a loss-in-weight twin screw feeder. As the material nears the end of the extruder, Hydromorphone HCl is fed by a second loss-in-weight twin screw feeder. This allows two materials with vastly different particle sizes to be compounded into a single, homogeneous mass. Additionally, Hydromorphone HCl is exposed to very little shear and heat. As the compounded mixture exits the extruder, the material is pelletized into a form that can be further processed.

Compounded pellets can then be transferred to an injection molding process to prepare the implants. In this process, the compounded pellets are heated until they become molten and are subsequently injected into a die that forms a central channel. In one embodiment, a second die is used to inject an impermeable coating such as neat EVA onto the implant.

### Results and Discussion

The viscosity of the matrix polymer must be sufficiently low in order to flow into a die. In order to determine the feasibility of various EVA grades for a product such as this, small scale formulations were prepared and tested on a Tinius Olsen melt plastometer.

Rather than using Hydromorphone HCl for initial experiments, Dextromethorphan HBr was used as the model drug as the particle size and solubility characteristics of these two compounds are very similar.

### Evatane® Selection

Grades of cryogenically ground EVA chosen for feasibility studies include: Evatane® 42-60, Evatane® 33-400, and Evatane® 28-800. In each case, EVA copolymers were mixed with Dextromethorphan HBr in a 1:1 ratio.

### Evatane 42-60

Evatane® 42-60 (42% vinyl acetate content, 60 g/10 min melt flow index) has properties very similar to that of Elvax® 40W. Evatane® 42-60 powder was blended with Dextromethorphan HBr in a polyethylene bag by hand for approximately 5 minutes. The resulting blend was placed in the Tinius Olsen melt plastometer and was allowed to equilibrate at 75.0° C. for 5-minutes. A 16.6 kg weight was used to press the melted blend through the 0.0810-inch orifice. At this temperature, a visual inspection of the extrudate confirmed that the viscosity of the mixture was too high to flow through the die. A visual inspection of the extrudate at 95° C. and 120° C. revealed that the composite mixture was very viscous and 16.6 kg was not enough weight to provide a constant flow. When the temperature of the plastometer was further increased to 130° C., the extrudate became less viscous and flowed from the plastometer. However, this temperature is likely too high and may cause degradation of Hydromorphone HCl.

### Evatane 33-400

Evatane® 33-400 (33% vinyl acetate content, 400 g/10 min melt flow index) powder was subjected to the same test as described above at temperatures of 65° C., 75° C., 95° C., and 110° C. A visual inspection of the resulting extrudates confirmed that the viscosity decreased as the temperature was increased. It was determined that the extrudate at 65° C. and 75° C. was too viscous to adequately flow into and fill a mold. At 95° C. and 110° C., the composite mixture was substantially less viscous and could potentially fill a mold.

### Evatane 28-800

A formulation containing Evatane® 28-800 (28% vinyl acetate content, 800 g/10 min melt flow index) was also prepared by the method described above. At 75.0° C., a visual inspection of the extrudate was performed and although it flowed through the die, it was determined that the viscosity was too high flow into and fill a mold. The experiment was repeated at a temperature of 95° C. and the viscosity of the extrudate was dramatically decreased. A pseudo disk shaped die was placed directly below the plastometer where the extrudate is expelled and allowed to fill. The die was evenly filled with the composite mixture and a disk was prepared. The viscosity and flow of the composite at 95° C. was comparable to that of the Evatane® 33-400 at 110° C.

### Prototype Fabrication

Based on results of the viscosity study, three grades of Evatane® were chosen for further studies: Evatane® 28-800, Evatane® 28-420, and Evatane® 33-400. Formulations containing Dextromethorphan HBr and EVA were evaluated on the Leistritz twin screw extruder and the prototype injection molding device. Dextromethorphan HBr was chosen as the model drug in order to develop processing conditions due to its cost relative to Hydromorphone HCl.

### Extrusion Process Development

Evatane® 28-800, 28-420, and 33-400 pellets were procured from Arkema for process development activities. Coiled feed screws were utilized such that Evatane® could be fed from the first feeder.

The Leistritz twin-screw extruder was set up to extrude powdered Evatane® 28-800 with downstream feeding of Dextromethorphan HBr. A composite extrusion screw was designed and installed such that minimal shear forces would be applied to the molten material. The extruder was equilibrated at a temperature of 80° C. prior to extrusion. Once equilibrated, the extruder was started at 300 rpm and each feeder was set to deliver 0.5 kg/hr.

The extrudate exited through a die with two 2-mm diameter holes spaced apart by 1-inch. The extrudate was found to exhibit a very low viscosity upon exiting the extruder. The two individual strands became intertwined, adhered to the conveyor, and exhibited erratic flow. The strands were cooled by forced air and subsequently pelletized. It was determined that the viscosity of the extrudate should be increased to prevent intertwining and adhering of the extrudate to the conveyor.

In order to optimize the extrusion process, steps were taken to increase the viscosity of the extrudate. This was accomplished by lowering the extrusion temperature to 60° C. and by reducing the extrusion speed to 100 rpm. At these conditions, the extrudate viscosity increased significantly and provided an acceptable product. The extrudates did not show any signs of intertwining or adhering to the conveyor belt. The strands were subsequently pelletized. Evatane® 28-800 was replaced with 33-400 and extruded at the same conditions with excellent results.

Coiled screws were obtained and Evatane® 28-420, 28-800, and 33-400 pellets were extruded with downstream feeding of granulated Dextromethorphan HBr. The extrusion screw speed for each grade of EVA was set at 160, 200, and 300, respectively. Each feeder was set the deliver 0.5 kg/hr and the extruder temperature was set at 55° C. for all three grades. These conditions produced excellent results.

### Prototype Injection Molding

In order to investigate the release profile of various sized disks, injection molds have been prepared such that the height and diameter of the disk varies 20% in each direction with the center channel held constant at 1.25 mm. Implant dimensions chosen for this study are shown in FIG. 2.

Additionally, the dissolution rate can be modulated by the polymer to drug ratio and size of the center channel.

For the manufacture of prototype implants, the Tinius Olsen melt plastometer was used as a bench top injection molder. Nine molds containing depressions with center channels have been fabricated to fit on the bottom of the melt plastometer to accept molten polymer.

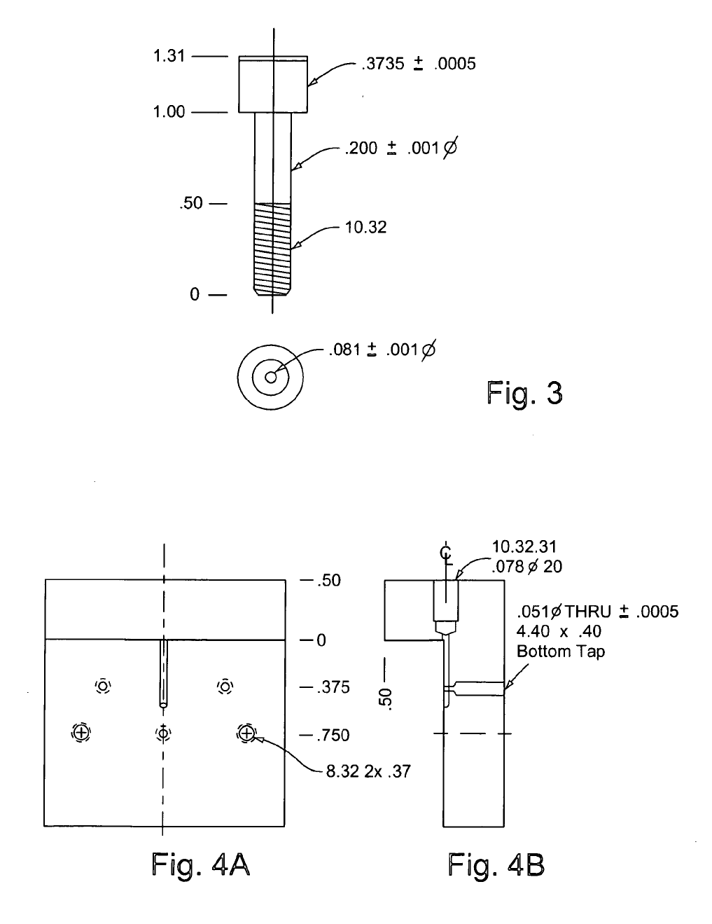

The injection nozzle that is used to transfer the molten polymer from the melt plastometer to the molds is shown in FIG. 3.

The nozzle contains an orifice with a diameter of 0.081-inches

The injection nozzle attaches to the mold base which is illustrated in FIGS. 4A and 4B. The injection base has pins with a 1.25 mm diameter that provide for central channels.

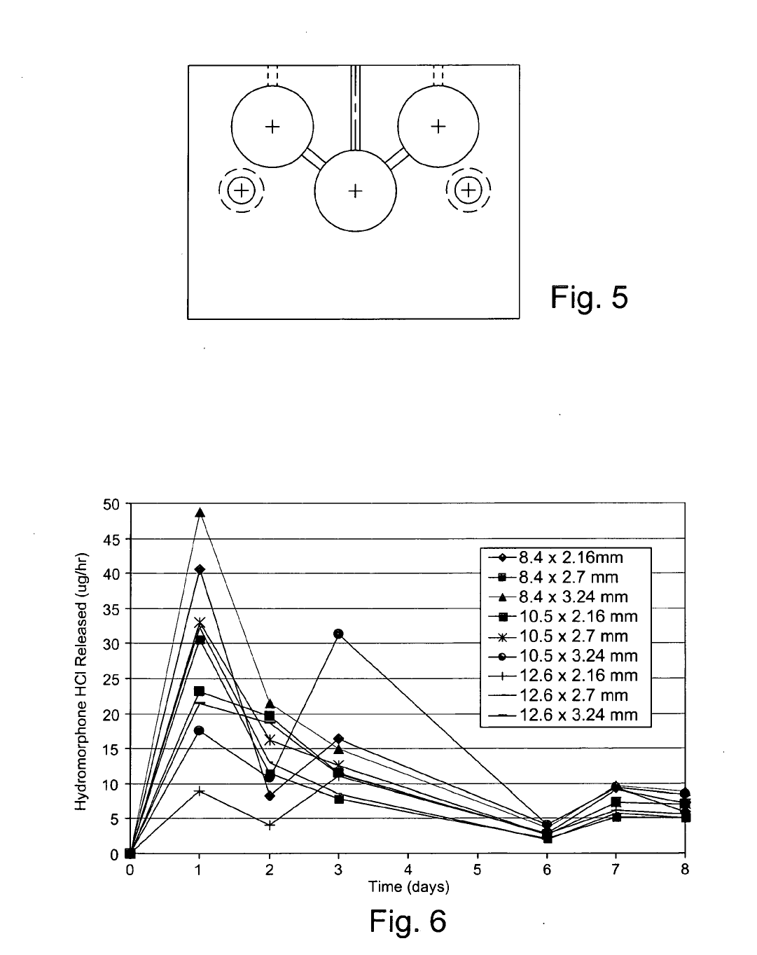

The injection base attaches to the injection mold (which forms the disks), which is illustrated in FIG. 5.

The injection mold contains disk shaped reservoirs with vents to allow air to escape. Once the injection base and injection mold are secured to each other, pins in the injection base are moved inward until they come into contact with the injection mold, which form a center channel.

Once the compounded polymers are sufficiently melted, weights are placed on top of a piston to force the composite mixture from the heated cylinder into the fabricated molds.

### Injection Molding Process Development

Compounded mixtures obtained from the extrusion process development activities were used to develop the injection molding process. Pellets containing equal amounts of Evatane® 28-800 and Dextromethorphan HBr were added to the extrusion plastometer and allowed to equilibrate for 5 minutes at 95° C. During the equilibration time, the nozzle was plugged and a total mass of 10.0 kg was used to compact the material. Once equilibrated, the mold, which was at room temperature, was placed onto the injection nozzle and a total mass of 20.6 kg was added to the piston. It was found that the composite mixture cooled upon leaving the injection nozzle and did not adequately fill the mold.

In order to address this issue, the equilibration temperature was increased to 105° C. and the mold was warmed to 75° C. on a hot plate. Once weight was added onto the piston, the polymer flowed freely into the mold. However, upon separating the mold from the base, it was discovered that the disks adhered slightly to the aluminum mold due to its surface characteristics. It was found that stearic acid provides sufficient lubrication to prevent disks from adhering to the molds. Additionally, the mold must be cooled to room temperature to ensure that the disks do not adhere to the mold.

A trial was conducted with compounded Evatane® 33-400. It was discovered that the disks containing this grade of Evatane® were significantly more difficult to remove from the mold. Ejection pins were added to each of the molds. It was found that retracting the pins and removing them from the die followed by cooling with compressed air is an effective method of removing the disks without imparting damage.

### Coating Process and Dissolution Analysis

Prior to performing dissolution studies, multiple polymers were tested as coating agents in order to determine which polymer could successfully impede the release of an active ingredient from the disk. Polymers tested included poly(methyl methacrylate) (PMMA), polyvinyl acetate (PVA), Ethocel® 100, cellulose acetate, and Evatane® 28-800. Most of these polymers were dissolved in a solvent such as acetone or ethanol and then used to dip coat disks. Some of the polymers were also mixed with hydrophobic plasticizers to increase the flexibility of the polymers. The Evatane® coating was applied using a hot-melt gun and a die rather than by solution. Each coating entirely covered the disk (including center channel) and was allowed to cool for an adequate amount of time before applying subsequent coatings.

The dissolution of Hydromorphone HCl or Dextromethorphan HBr from prototype implants was measured by the method previously employed by Axxia Pharmaceuticals. In this dissolution method, disks are placed in scintillation vials with 10 mL of 0.1 M pH 7.4 phosphate buffer. The scintillation vials were placed in an oven with a temperature set point of 37° C. For the initial tests, dissolution media was only removed once after 16-24 hours to determine if the release of the active drug was impeded.

A summary of the coating solutions and results can be seen in Table 1.

TABLE 1
Coating agents, conditions, and results for implantable disks

| | | | Blocked | |
|---|---|---|---|---|
| Polymer | Plasticizer | Solvent | Release? | Comments |
| 10% PMMA | n/a | Acetone | No | Brittle coating, |
| | | | | Numerous air bubbles |
| | | | | in coating |
| 25% PMMA | n/a | Acetone | No | Smooth coating, Few |
| | | | | air bubbles in coating |
| 19% PMMA | 1% DEP | Acetone | No | Smooth coating, Few |
| | | | | air bubbles in coating |
| 10% PVA | n/a | Acetone | No | Smooth coating |
| 15% PVA | n/a | Acetone | No | Smooth coating, |
| | | | | Aesthetically pleasing |
| 5% Ethocel 100 | n/a | Ethanol | Not Tested | Many air bubbles in coating |
| 13.3% Cellulose | n/a | Acetone | No | Disk swollen, Buffer |
| Acetate | | | | diffused between |
| | | | | coating and disk |
| 12.1% Cellulose | 1.5% Triacetin | Acetone | No | Disk swollen, Buffer |
| Acetate | | | | diffused between |
| | | | | coating and disk |
| Evatane 28-800 | n/a | n/a | Yes | Very flexible coating, |
| | | | | Blocked release of |
| | | | | Dextromethorphan HBr |
| | | | | and Hydromorphone HCl |

Evatane® 28-800 was the only coating agent that completely prevented the release of Hydromorphone HCl and Dextromethorphan HBr from the implant after 16-24 hours in 10 mL of 0.1 M pH 7.4 phosphate buffer at 37° C. Thus, the nine initial disk sizes were coated with Evatane® 28-800 and have a center channel in both the disk and the coating.

### Dissolution Results

### Unmicronized Hydromorphone Hydrochloride

Unmicronized Hydromorphone Hydrochloride was used for to prepare disks in initial studies. 80% of the unmicronized Hydromorphone Hydrochloride has a particle size of less than 75 microns.

### Disk Size and Evatane® Grade Study

Samples were prepared containing 50.0% Hydromorphone Hydrochloride and 50.0% Evatane® 28-800 by the method outlined above in Injection Molding Process Development. Sets of samples were prepared (n=3), as described above in Prototype Injection Molding, with the nine dimensions as outlined in order to investigate the affect of different disk dimensions on the dissolution rate of Hydromorphone Hydrochloride. Additionally, discs containing a different grade of Evatane® were also prepared. Three disks composed of 50% Evatane® 28/420 and 50% Hydromorphone Hydrochloride and three disks composed of 50% Evatane® 33/400 and 50% Hydromorphone Hydrochloride with a disk size of 12.6×2.7 mm were prepared. All eleven sets of three disks each were coated with Evatane® 28/800 as described above in Coating Process and Dissolution Analysis.

Coated disks where examined under a Leica EZ4D Stereoscope in order to determine if the coating and center channel were acceptable for dissolution studies. Any air bubbles or abnormalities in the coating were removed and patched with a soldering gun and a hot-melt gun.

All the disks were attached to sinkers and placed in scintillation vials with 10 mL of 0.1 M pH 7.4 phosphate buffer at 37° C. Buffer solution was removed and replaced at t=1, 2, 3, 6, 7, and 8 days. The amount of Hydromorphone Hydrochloride that was released from each of the nine sized disks containing Evatane® 28-800 disks is shown in the graph of FIG. 6. This graph of FIG. 6 shows that by Day 8 the release of Hydromorphone Hydrochloride from all nine dimensions of disks is well below the target release rate of approximately 4.0 mg/day (166.7 ug/hr). In addition, an unexpected initial burst release is seen in almost all samples.

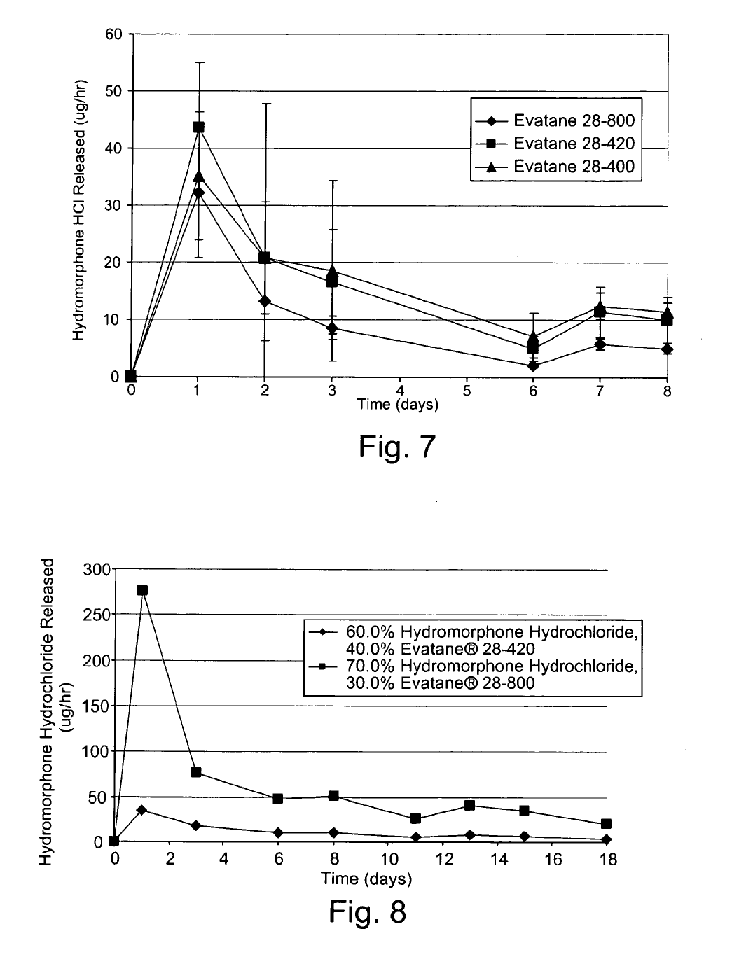

The amount of Hydromorphone Hydrochloride that was released from each of the three disks with different grades of Evatane® is shown in the graph of FIG. 7. The graph of FIG. 7 shows that the grade of Evatane® used as the polymer matrix does not affect the release rate of Hydromorphone Hydrochloride. In addition, an unexpected initial burst release is again seen in these samples.

### Increased Drug Loading Study

In order to increase the release rate of Hydromorphone Hydrochloride from the disks to achieve the target release rate of approximately 4.0 mg/day, the concentration of Hydromorphone Hydrochloride within each disk was increased.

Samples were prepared containing 60.0% Hydromorphone Hydrochloride and 40.0% Evatane® 28-420, 70.0% Hydromorphone Hydrochloride and 30.0% Evatane® 28-800, and 60.0% Hydromorphone Hydrochloride and 30.0% Evatane® 28-420 and 10.0% Polyethylene Glycol 4000 by the method outlined above.

Additional samples containing 70.0% Hydromorphone Hydrochloride and 30.0% Evatane® 28-420 as well as samples with 70.0% Hydromorphone Hydrochloride and 20.0% Evatane® 28-800 and 10.0% Polyethylene Glycol 4000 were attempted, but were abandoned due to the inability to extrude and the brittleness of formed disks, respectively.

Sets of samples were prepared (n=3), as described above (Prototype Injection Molding), with the 12.6×2.7 mm dimension in order to investigate the affect of increased drug loading on the dissolution rate of Hydromorphone Hydrochloride. All three sets of three disks were coated with Evatane® 28-800 as described above (Coating Process and Dissolution Analysis) and one additional 60.0% Hydromorphone Hydrochloride and 40.0% Evatane® 28-420 was completely coated (including the center channel) to act as a control.

Coated disks where examined under a Leica EZ4D Stereoscope in order to determine if the coating and center channel were acceptable for dissolution studies and within the required specifications. Any air bubbles or abnormalities in the coating were removed and patched with a soldering gun and a hot-melt gun.

All disks were attached to sinkers and placed in scintillation vials with 10 mL of 0.1 M pH 7.4 phosphate buffer at 37° C. Buffer solution was removed and replaced at t=1, 3, 6, 8, 11, 13, 15, and 18 days. The amount of Hydromorphone Hydrochloride that was released from the 60.0% Hydromorphone Hydrochloride with 40.0% Evatane® 28-420 and 70.0% Hydromorphone Hydrochloride with 30.0% Evatane® 28-800 is shown in the graph of FIG. 7. The graph of FIG. 7 shows that by Day 3 the release of Hydromorphone Hydrochloride from both types of disks is well below the target release rate of approximately 4.0 mg/day (166.7 ug/hr). In addition, an unexpected initial burst release is seen in both samples.

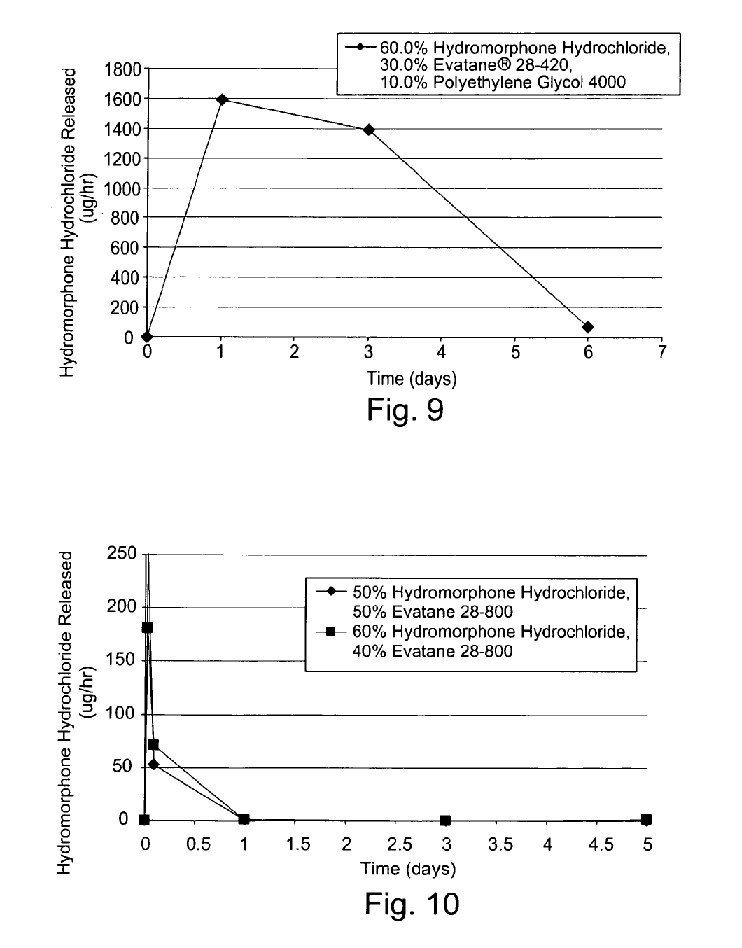

The amount of Hydromorphone Hydrochloride that was released from the 60.0% Hydromorphone Hydrochloride and 30.0% Evatane® 28-420 and 10.0% Polyethylene Glycol 4000 disks is shown in the graph of FIG. 9. The dissolution of these samples was stopped after 6 days due to the very high release rate of Hydromorphone Hydrochloride. The high release rate from this disk is most likely due to cracks within the disk structure. Polyethylene Glycol 4000 caused the disks to become very brittle and due to the handling of the disks, cracks were most likely formed during the removal of the disks from the injection molds or during the coating process.

The control disk showed no release of Hydromorphone Hydrochloride during the eighteen days in dissolution buffer, confirming previous studies which showed that Evatane® blocks the release of drug from the matrix.

### Micronized Hydromorphone Hydrochloride

It was hypothesized that micronizing Hydromorphone Hydrochloride may eliminate the burst effect seen with unmicronized Hydromorphone Hydrochloride as well increase the dissolution rate by forming more channels within the carrier matrix. Hydromorphone Hydrochloride was micronized using a Hosokawa Alpine 50 AS Spiral Jet Mill System. The average particle size was reduced approximately tenfold to about 5 microns.

### Drug Loading Study

A blend containing 65% micronized Hydromorphone Hydrochloride and 35% Evatane® 28-800 was mixed and loaded into the melt plastometer. The blend was allowed to equilibrate at temperatures as high as 140° C., but the blend failed to extrude through the orifice. It is obvious that micronized Hydromorphone Hydrochloride changes the rheology of the extrudate due to the increased surface area. Thus, the concentration of micronized Hydromorphone Hydrochloride was decreased to form acceptable extrudate.

Samples were prepared containing 50.0% Hydromorphone Hydrochloride with 50.0% Evatane® 28-800 and 60% Hydromorphone Hydrochloride with 40% Evatane 28-800 by the method outlined above. These blends were successfully extruded and the molding of disks was attempted as described above. Due to the rheological changes in the extrudate, the molds experienced incomplete filling and multiple air pockets were observed in each disk.

An alternative method for filling molds was explored. The injection base and injection mold were both lubricated with stearic acid and placed on a hot plate with a temperature of 150-200° C. Pelletized extrudate was placed within the injection mold until and manipulated until the two outside reservoirs were filled with composite material. The injection base and injection mold are then fastened together and the pins in the injection base are moved inward until they come into contact with the injection mold, which form a center channel. The mold was removed from the hot plate and cooled to room temperature. Three disks with a size of 10.5×2.7 mm of each concentration were obtained and both sets were coated with Evatane® 28-800 as described above.

Coated disks were examined under a Leica EZ4D Stereoscope in order to determine if the coating and center channel were acceptable for dissolution studies and within the required specifications. Any air bubbles or abnormalities in the coating were removed and patched with a soldering gun and a hot-melt gun. Disks were cured in an oven at 50° C. in order to ensure that the disk was properly adhered to the disk.

All the disks were attached to sinkers and placed in scintillation vials with 10 mL of 0.1 M pH 7.4 phosphate buffer at 37° C. Buffer solution was removed and replaced at t=30 min, 2 hr, and 1, 3, and 5 days. The amount of Hydromorphone Hydrochloride that was released from both sets of disks is shown in FIG. 10. The graph of FIG. 10 shows that within 2 hours the release of Hydromorphone Hydrochloride from both types of disks is well below the target release rate of approximately 4.0 mg/day (166.7 ug/hr). The release rate almost completely shuts down by the Day 1 time point. In addition, an undesired initial burst release is seen in both samples that is likely due to Hydromorphone Hydrochloride on the surface of the inside channel.

### Scanning Electron Microscope (SEM) Images

A scanning electron microscope (SEM) was used on disks containing unmicronized and micronized Hydromorphone Hydrochloride in order to obtain information about various samples' surface topography and composition.

### Unmicronized Hydromorphone Hydrochloride

Samples containing 60.0% Hydromorphone Hydrochloride and 40.0% Evatane® 28-420 which were placed in 0.1 M pH 7.4 phosphate buffer at 37° C. were examined with the SEM. The pictures showed good annealing between the coating and the composite disc. Another picture showed the pores and channels formed by the dissolution of Hydromorphone Hydrochloride from the Evatane® matrix. This image showed that open channels were formed without the entrapment of Hydromorphone Hydrochloride.

### Micronized Hydromorphone Hydrochloride

Samples containing 50.0% micronized Hydromorphone Hydrochloride with 50.0% Evatane® 28-800 which were not exposed to any dissolution media and samples containing 60% micronized Hydromorphone Hydrochloride with 40% Evatane 28-800 which were placed in 0.1 M pH 7.4 phosphate buffer at 37° C. were examined with the SEM. The images clearly showed air pockets and pores formed from the processing of these discs without the use of the Tinius Olsen melt plastometer. The center channel of this disk had minimal exposure of micronized Hydromorphone Hydrochloride particles, thus inhibiting the release of drug. The inside matrix of the disk had many visible micronized Hydromorphone Hydrochloride particles, but may be below the percolation threshold which may inhibit their release. Another image showed minimal exposure of micronized Hydromorphone Hydrochloride particles on surfaces in contact with the mold. The lack of Hydromorphone Hydrochloride particles on the surface of the disk may be due to skinning of the Evatane® polymer.

Another image showed a cross section of the tested 60.0% micronized Hydromorphone Hydrochloride with 40.0% Evatane® 28-800 discs. This picture showed good annealing between the coating and the composite disk. A further image showed a cross section of the inside channel as well as the inner matrix of the disc. The center channel of this disk had no formed channels or pores and thus drug could not be released from the disc. The inside of the disk had many visible micronized Hydromorphone Hydrochloride particles. As previously stated, the lack of Hydromorphone Hydrochloride particles on the surface of the disk may be due to skinning of the Evatane® polymer during processing.

### Extrusion of Elasthane™

An alternative polymer, Elasthane™, a human implant grade aromatic polyether type thermoplastic polyurethane was also tested. Elasthane™ thermoplastic polyether urethane is produced by The Polymer Technology Group and is approved to be used in implant medical devices for longer than 30 days. This polymer is available in three grades. Elasthane™ 80A was selected for feasibility studies due to its relatively low melt index of the three available grades and because it has the lowest recommended optimum extrusion temperature of 171-197° C.

The Leistritz twin-screw extruder was set up to extrude Elasthane™. Since Elasthane™ is only available in a pellet form, coiled screws were used in the feeder. The same composite extrusion screw was designed and installed as used with Evatane® polymers, such that minimal shear forces would be applied to the molten material. The extruder was equilibrated at a temperature of 180° C. prior to extrusion. Once equilibrated, the extruder was started at 50 rpm and the feeder was set to deliver 0.5 kg/hr of polymer.

At first, no die was attached to the extruder and the extrudate was found to be transparent and fairly viscous. The temperature of the extruder was decreased to 170° C. and the viscosity of the extrudate increased while it remained transparent. The temperature was then raised to 190° C. and a substantially less viscous, transparent, very elastic extrudate was formed. The screw speed was increased to 75 rpm and a 6.25 mm single round bore die was attached to the extruder. Rods were formed without pulsing from the die. This resin was selected because it can be extruded and molded at a temperature below the decomposition point of the opiod.

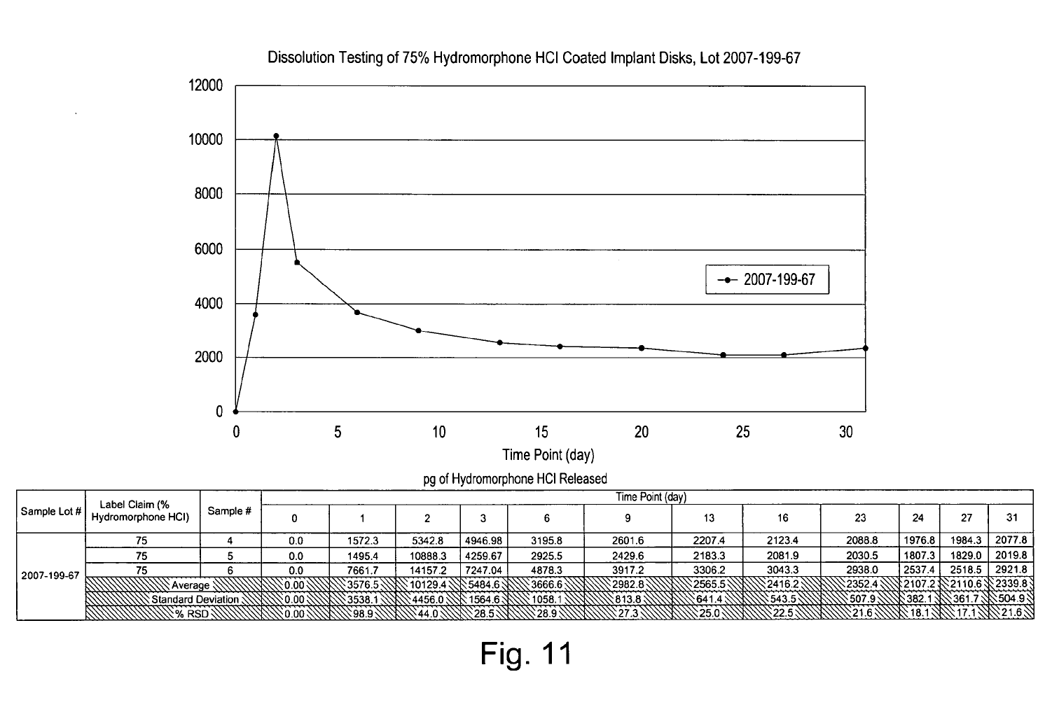

Implants which were altered from the above described implants by producing the central channel by mechanical means (perforation or drilling) were also tested. The plot of FIG. 11 shows the dissolution profile of these implants to the 31 day time point.

It will be readily apparent to those skilled in the art that numerous modifications and additions may be made to the present invention, the disclosed device, and the related system without departing from the invention disclosed.

1. A subcutaneous delivery system comprising:
i) a biocompatible thermoplastic polymer matrix,
ii) a therapeutic agent embedded homogeneously in said matrix, and
iii) a biocompatible drug impermeable thermoplastic polymer coating said matrix,
wherein said delivery system has a geometry such that there is an external coated wall and an internal uncoated wall forming an opening for release of said therapeutic agent, and the distance between the uncoated wall and the coated wall opposite the uncoated wall is substantially constant throughout the delivery system.

2. A subcutaneous delivery system as in claim 1, wherein said delivery system is cylindrical in shape.

3. A subcutaneous delivery system as in claim 1, wherein said matrix is EVA.

4. A subcutaneous delivery system as in claim 1, wherein said matrix is urethane.

5. A subcutaneous delivery system as in claim 1, wherein said matrix and coating are non-biodegradable.

6. A subcutaneous delivery system as in claim 1, wherein said matrix and coating are biodegradable.

7. A subcutaneous delivery system as in claim 1, wherein said therapeutic agent is an opiod.

8. A subcutaneous delivery system as in claim 1, wherein said therapeutic agent is selected from the group consisting of hydromorphone, etorphine and dihydroetorphine.

9. A subcutaneous delivery system as in claim 1, wherein said coating is EVA.

10. A subcutaneous delivery system as in claim 1, wherein said coating is urethane.

11. A subcutaneous delivery system as in claim 1, wherein said coating contains one or more inter-laminar diffusional drug barrier layers or films based on homopolymers of vinylidene chloride or copolymers of vinylidene chloride and vinyl chloride.

12. A subcutaneous delivery system as in claim 1, wherein said coating contains an adhesive tie coat between said coating and polymer matrix.

13. A subcutaneous delivery system as in claim 12, wherein said tie coat is an ethylenic anhydride either blended together with a different ethylinic anhydride or blended with an ethylenic copolymer, a copolyester, a Nylon copolymer or a thermoplastic polyurethane.

14. A subcutaneous delivery system as in claim 1, wherein said coating is two layers.

15. A subcutaneous delivery system as in claim 1, wherein said coating is three layers.

16. A subcutaneous delivery system as in claim 1, further comprising an outer coating having a second polymer matrix containing a second therapeutic agent.

17. A subcutaneous delivery system as in claim 14, wherein each coating is 24-48 microns thick.

18. A subcutaneous delivery system comprising
i) an EVA polymer matrix,
ii) a therapeutic agent embedded homogeneously in said matrix,
iii) a biocompatible drug impermeable EVA polymer coating said matrix
wherein said delivery system has a geometry such that there is an external coated wall and an internal uncoated wall forming an opening for release of said therapeutic agent, and the distance between the uncoated wall and the coated wall opposite the uncoated wall is substantially constant throughout the delivery system.

19. A subcutaneous delivery system comprising
i) a biocompatible thermoplastic urethane polymer matrix,
ii) a therapeutic agent embedded homogeneously in said matrix,
iii) a biocompatible drug impermeable thermoplastic urethane polymer coating said matrix,
wherein said delivery system has a geometry such that there is an external coated wall and an internal uncoated wall forming an opening for release of said therapeutic agent, and the distance between the uncoated wall and the coated wall opposite the uncoated wall is substantially constant throughout the delivery system.

20. A method of providing prolonged relief of pain in a mammal suffering from pain comprising subcutaneously administering the subcutaneous delivery system of claim 8.

21. A method of producing a subcutaneous implant comprising the steps of:
i) forming a matrix polymer sheet by hot melt compounding a first thermoplastic polymeric resin with a therapeutic agent,
ii) die cutting said sheet to form polymer matrix, and
iii) coating said polymer matrix with a second thermoplastic polymeric resin.

22. A method as in claim 21 wherein prior to step i) is the step of dry blending said first thermoplastic polymeric resin with a therapeutic agent.

23. A method as in claim 21 wherein after step iii) is the step of drying coated polymer matrix.

24. A method as in claim 21 wherein after step iii) is the step of forming a channel in the coated polymer matrix.

25. A method as in claim 21 wherein said first thermoplastic polymeric resin is a resin blend.

26. A method as in claim 21 wherein said second thermoplastic polymeric resin is a resin blend.

27. A method as in claim 21, wherein said coating said matrix polymer is done by solution coating.

28. A method as in claim 21, wherein said coating said matrix polymer is done by hot melt extrusion.

29. A method as in claim 21, wherein said coating said polymer matrix is done by powder coating and then thermal fusion.

30. A method as in claim 21 wherein more than one coating is applied to said polymer matrix.

31. A method as in claim 30 wherein an outer coating is a second polymeric matrix containing a second therapeutic agent.

32. A method of producing a subcutaneous implant delivery system comprising the steps of:
i) hot melt extrusion of a first thermoplastic polymeric resin with a therapeutic agent to form a polymer matrix in a cylindrical shape,
ii) powder coating and thermal fusing a second thermoplastic polymeric resin on said polymer matrix to form a therapeutic agent impermeable coating, and
iii) forming an uncoated channel in said implant.

33. A method of producing a subcutaneous implant delivery system having an uncoated central channel comprising the steps of:
co-extruding of a first thermoplastic polymeric resin and a therapeutic agent and a second thermoplastic polymeric resin into a multiple cavity die to form a coated polymer matrix.

34. A method as in claim 33 wherein said uncoated central channel is formed in the hot melt co-extrusion process.

35. A method as in claim 33 wherein said uncoated central channel is formed after the coated polymer matrix is formed.

36. A method as in claim 21 wherein said first thermoplastic polymeric resin is extruded with a foaming agent.
